(19) United States
(12) Patent Application Publication
(10) Pub. No.: US 2021/0322696 A1
(43) Pub. Date: Oct. 21, 2021

(54) VENTILATOR

(71) Applicant: Honeywell International Inc., Charlotte, NC (US)

(72) Inventors: Alex Harrison, Dorset (GB); Nicholas Withers, Somer (GB); Peter James Hedges, Dorset (GB); Robert Lings, Dorset (GB); Alexander Fell, Somer (GB); Carl Hall, Somer (GB); Mark Jeffrey Rayson, Somer (GB)

(21) Appl. No.: 17/234,360

(22) Filed: Apr. 19, 2021

Related U.S. Application Data
(60) Provisional application No. 63/012,761, filed on Apr. 20, 2020.

(51) Int. Cl. A61M 16/00; A61M 16/08; A61M 16/12; A61M 16/20

(52) U.S. Cl. CPC A61M 16/022; A61M 16/0003; A61M 16/0816; A61M 2202/0208; A61M 16/12; A61M 16/201; A61M 2016/0027; A61M 16/009

(57) ABSTRACT

In some examples, a ventilator includes a pneumatic connection configured to receive supply gas; an inspiratory path configured to deliver conditioned gas to lungs of a patient; a valve pneumatically connected to the pneumatic connection and configured to allow flow of the supply gas to the inspiratory path when the valve is open and block flow of the supply gas to the inspiratory path when the valve is closed; a pressure sensor configured to measure a pressure of the supply gas at the pneumatic connection; and electronic control circuitry configured to control an opening and closing of the valve based on the measured pressure to produce a desired volume of conditioned gas in the inspiratory path.

This application claims the benefit of U.S. Provisional Patent Application 63/012,761, filed Apr. 20, 2020, the entire content of which is incorporated herein by reference.

### TECHNICAL FIELD

This disclosure relates to ventilators.

### BACKGROUND

A ventilator is a machine that mechanically aids a patient in breathing by moving a conditioned gas into and out of one or more lungs of the patient. Many ventilators, particularly those found in modern hospitals, have electronic controls that allow a clinician to set various parameters related to the delivery of the conditioned gas to the patient.

### SUMMARY

In examples described herein, a ventilator is configured to provide a desired volume of conditioned gas to an inspiratory path by at least controlling an opening and closing of a valve based on a measured pressure of supply air. The conditioned gas may be delivered to lungs of a patient via the inspiratory path. As will be described in more detail below, example ventilators described herein are configured to choke the flow of conditioned gas into an inspiratory path and determine tidal volume as a function of time, which may reduce component complexity and thus may enable ventilators to be manufactured at scale and deployed in relatively short time frames compared to more complex ventilators.

According to one example, a ventilator includes a pneumatic connection configured to receive supply gas; an inspiratory path configured to deliver conditioned gas to lungs of a patient; a valve pneumatically connected to the pneumatic connection and configured to allow flow of the supply gas to the inspiratory path when the valve is open and block flow of the supply gas to the inspiratory path when the valve is closed; a pressure sensor configured to measure a pressure of the supply gas at the pneumatic connection; and electronic control circuitry configured to control an opening and closing of the valve based on the measured pressure to produce a desired volume of conditioned gas in the inspiratory path.

According to another example, a method includes receiving a supply gas at a pneumatic connection; measuring a pressure of the supply gas at the pneumatic connection; and delivering, via an inspiratory path, conditioned gas to lungs of a patient, wherein delivering the conditioned gas to the lungs of the patient comprises: for a first measured pressure of the supply gas at the pneumatic connection, increasing an amount of time which a valve is opened to increase a volume of the conditioned gas delivered to the lungs of the patient during a first respiratory cycle; and for a second measured pressure of the supply gas at the pneumatic connection, decreasing the amount of time which the valve is opened to decrease a volume of the conditioned gas delivered to the lungs of the patient during a second respiratory cycle.

The details of one or more examples are set forth in the accompanying drawings and the description below. Other features, objects, and advantages will be apparent from the description, drawings, and claims.

### BRIEF DESCRIPTION OF DRAWINGS

FIG. 1 shows a conceptual diagram of an example ventilator in accordance with the techniques of this disclosure.

FIG. 2 shows a conceptual diagram of an example ventilator in accordance with the techniques of this disclosure.

FIG. 3 shows an example of controls that a user may interact with to set the operating parameters for a ventilator in accordance with the techniques of this disclosure.

FIG. 4 shows a graphical representation of an example of an inspiratory and expiratory cycle for a specific set of operating parameters.

FIG. 5 shows a conceptual diagram of an example ventilator in accordance with the techniques of this disclosure.

FIG. 6 represents an example process that can be performed by a ventilator of this disclosure.

### DETAILED DESCRIPTION

The 2020 Coronavirus disease (COVID-19) Pandemic has caused a large number of hospitalizations globally due to COVID-19-related respiratory failure, which has resulted in large numbers of patients requiring mechanical ventilatory support. Demand for medical ventilators has overwhelmed the capacity of health care systems and existing supply chains worldwide.

One way to address an urgent demand for mechanical ventilators is through Emergency Use Ventilators (EUVs) that can be produced at scale and by drawing on diverse supply chains. EUVs can be designed to provide a clinically acceptable level of performance and functionality for a subset of patients, which enables the design to be greatly simplified relative to a more state of the art modern ventilator. As one example, while some ventilators may be configurable to be used with patients of all ages and sizes, an EUV may be relatively less configurable, such that the EUV cannot be used with certain segments of the population, such as children or people who are severely overweight.

Ventilators are configured to receive supply gas from a gas supply, such as those found in hospitals, and produce, from the supply gas, a conditioned gas for a patient to breathe. The supply gas may, for example, be 100% oxygen or other gas with a high oxygen concentration. From the supply gas, the ventilator produces conditioned gas for a patient to breathe. The conditioned gas may have the same oxygen concentration as the supply gas or be mixed with air and have a reduced oxygen concentration relative to the supply gas. The conditioned gas may also be at a desirable pressure and limited to a desirable volume, as determined based on user inputs.

As will be described in more detail below, ventilators that are configured to choke the flow of conditioned gas into an inspiratory path and determine tidal volume as a function of time. The ventilator designs described herein may reduce component complexity and thus may enable EUVs to be manufactured at scale and deployed in relatively short time frames compared to more complex ventilators. This disclosure describes various techniques for building low cost, clinically useful, ventilators that can be produced using largely off the shelf components. The ventilators of this disclosure may also be designed to have a rugged form factor, such that the ventilators can better withstand transport. The ventilators described herein can potentially help make life saving mechanical ventilation widely available at a price point that enables more lives to be saved through rapid deployment.

A ventilator of this disclosure may include a relatively rugged physical enclosure containing a number of two and three port pneumatic solenoid valves which communicate via interconnecting pneumatic assemblies. The enclosure may have an external 100% oxygen or blended gas inlet configured to connect to a gas supply, such as a standard hospital oxygen supply. A ventilator of this disclosure may also include a port in the enclosure which allows ambient air to be drawn in to provide an air mix function and to provide an over pressure vent pathway out of the unit under specific conditions.

A ventilator of this disclosure may also include an outlet connection port configured to deliver the conditioned gas to the external patient circuit and an expiratory return connection port to receive the air exhaled by the patient. A ventilator of this disclosure may be configured to be compatible with the same external hoses and piping used in existing hospital ventilators. That is, a ventilator of this disclosure may receive gas from an oxygen supply and deliver conditioned gas to a patient using the same piping and tubing as existing ventilators.

A ventilator of this disclosure may be configured to receive electrical power, via a main power lead, from a standard electrical socket, such as 100-120 volt (V) U.S. socket, a 220-240V European socket, or any other such electrical power source. The ventilator may also include a backup source, such as a rechargeable battery, to provide continued use in the event of a main power outage.

A ventilator of this disclosure may also include a relatively simple visual display to inform the user of the instantaneous unit outlet pressure and the current peak setting and positive end-expiratory pressure (PEEP) settings, as well as visual indicators of over pressure, loss of power, failure to cycle, and various unit status warnings. The ventilator may alternatively or additionally be configured to produce audible indicators of over pressure, loss of power, failure to cycle, and various unit status warnings.

A ventilator of this disclosure may be configured to provide synchronized, volume controlled mandatory ventilation to a patient using an inspiratory solenoid which provides a constant flow of gas to the patient. An electronic controller (including control circuitry) of the ventilator may be configured to control the duration for which flow is delivered to the patient to provide a range of user adjustable tidal volumes which are available across a range of separate user adjustable Respiration Rates. The electronic circuitry may, for example, be implemented in analog-based electronics.

A ventilator of this disclosure potentially avoids the need for an internal pressure regulator, thus reducing complexity of the design. A ventilator of this disclosure can use electronic control algorithm to compensate for differences in supply pressure, within a specified range, by varying the duration of opening of a valve (e.g., the inspiratory solenoid) automatically based on feedback from an inlet pressure transducer. That is, in lieu of an expensive pressure regulator, a ventilator of this disclosure may have a constant, or near constant, flow volume, and thus use timing to control pressure and tidal volume.

An outlet pressure of the ventilator may be controlled based on feedback from a pressure sensor (e.g., a pressure transducer). If the pressure exceeds a peak pressure setting, then the ventilator may curtail an inspiratory cycle, meaning that the requested tidal volume will not be delivered at the unit outlet. In some examples, in such an instance, the ventilator may produce a visual or audible alert in response to the curtailing of the inspiratory cycle, the detection of a pressure exceeds the user peak pressure setting, or any combination thereof.

A ventilator of this disclosure may be configured to control a PEEP pressure against which the patient exhales using an expiratory solenoid that is controlled based on pressure feedback from an expiratory pressure transducer located near the expiratory return connection of the ventilator.

The ventilator may include a user interface that enables the user to select a range of peak and PEEP pressures for delivery at the outlet of the ventilator. The ventilator may be configured such that the peak and PEEP pressure settings are directly ganged or linked in a way that the peak pressure limit is set at a fixed, selectable amount, e.g., 15 cmH2O, 20 cmH2O, or 25 cmH2O, above the PEEP. A ventilator of this disclosure may also feature a mechanical relief valve to limit the pressure.

A ventilator of this disclosure may include a jet pump arrangement to entrain ambient air which allows a binary choice of concentrations of oxygen to be delivered. The binary choice may, for example, be approximately 50% oxygen or approximately 100% oxygen. The flow for both concentrations may be controlled by a common inspiratory solenoid. The jet pump arrangement may utilize a choked flow to achieve the constant, or near constant flow volume, which as introduced above, enables the ventilator to control tidal volume based on timing rather than with a pressure regulator.

A ventilator of this disclosure may also be configured to sense a depression in the expiratory pressure and automatically trigger a new inspiratory cycle if the patient attempts to take an early breath.

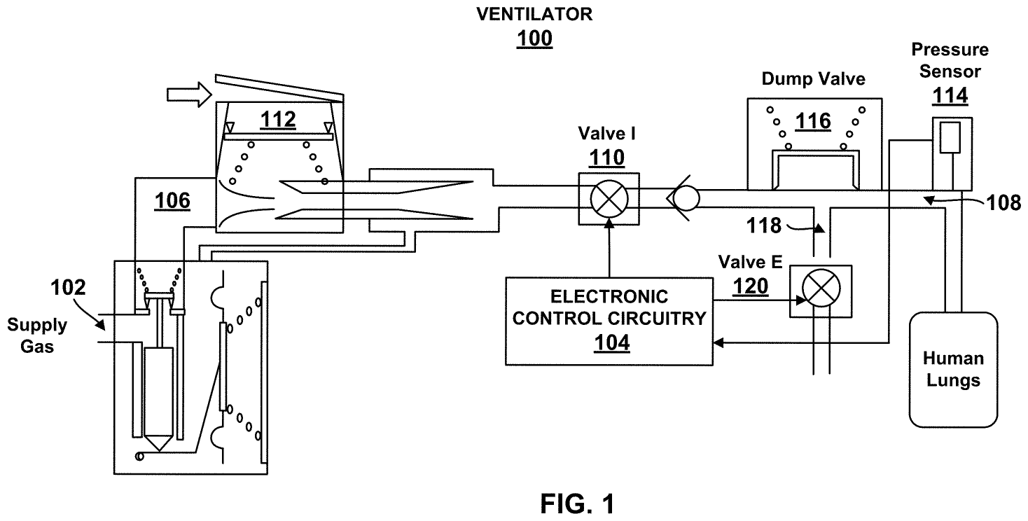

FIG. 1 shows a conceptual diagram of an example ventilator in accordance with the techniques of this disclosure. Ventilator 100 includes pneumatic connection 102, electronic control circuitry 104, supply path 106, inspiratory path 108, inspiratory valve 110 (Valve I 110 in FIG. 1), pressure sensor 114, dump valve 116, expiratory path 118, and expiratory valve 120 (Valve E 120 in FIG. 1).

A user, such as a doctor, nurse, or other medical clinician, may connect ventilator 100 to a gas supply via a pneumatic connection 102. Pneumatic connection 102 may, for example, include piping and/or tubing configured to define a pathway for gas from the gas supply into ventilator 100. Pneumatic connection 102 may, for example, be configured to allow the pressurised supply gas to flow without leaking. Pneumatic connection 102 may include a connector and an internal passage to allow the gas to flow to a balanced demand valve (BDV).

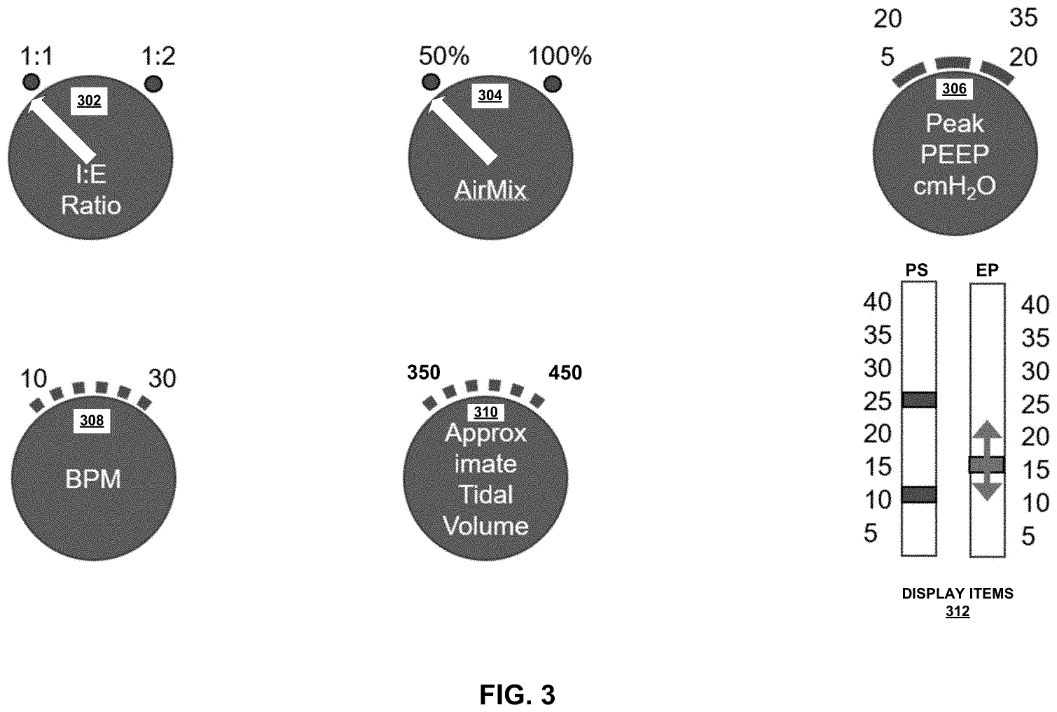

Electronic control circuitry 104 may include, or be coupled to, a user interface that allows a user of ventilator 100 to set operating parameters for ventilator 100. Electronic control circuitry 104 may, for example, be a variable-frequency-variable-duty-cycle electronic control circuit that forms the basis of the control of ventilator 100. Electronic control circuitry 104 can provide the timing framework which is used to deliver conditioned gas flow to the lungs of a patient. Examples of the operating parameters that may be controlled by a user will be described in more detail below with reference to FIG. 3.

Ventilator 100 is configured to deliver conditioned gas to the lungs of a patient via supply path 106 and inspiratory path 108. Electronic control circuitry 104 is configured to control the opening and closing of inspiratory valve 110 at a specific frequency to regulate the amount of conditioned gas being supplied to the patient and to control a “breaths per minute” being supplied to the patient (human lungs of the patient are schematically shown in FIG. 1) via inspiratory path 108. Generally speaking, a longer open interval for inspiratory valve 110 results in ventilator 100 delivering to a patient a larger volume of conditioned air, and more open intervals (e.g., more open intervals per minute) results in ventilator 100 delivering more breaths per minute. Electronic control circuitry 104 may alter the amount of conditioned gas being supplied and the number of breaths per minute being supplied by ventilator 100 by varying the open-close pattern of inspiratory valve 110. The volume of conditioned gas being driven into the patient may be selected by a user and regulated by electronic control circuitry 104.

As will be explained in more detail below, electronic control circuitry 104 may be configured to open inspiratory valve 110 for a period of time controlled by timing circuitry of electronic control circuitry 104 to achieve a desired inspiration volume. The time for which inspiratory valve 110 is held open, which may be referred to as an inspiration period, may depend on the pressure of the gas supply at pneumatic connection 102. Likewise, the time for which inspiratory valve 110 is held closed, which may be referred to as an expiration period, may depend, at least partially, on the pressure of the gas supply at pneumatic connection 102.

Inlet 112 is configured to be opened and closed to allow ambient air to mix with the supply gas. Generally, mixing ambient air with the supply gas produces a conditioned gas with a reduced oxygen concentration relative to the supply gas.

Inspiratory path 108 includes pressure sensor 114 which is configured to determine a pressure in inspiratory path 108. Supply path 106 may have an outlet nozzle leading into inspiratory path 108 that is sufficiently small, such that a pressure ratio across the nozzle results in a choked flow through the nozzle. In some examples, this choked flow may be achieved, for example, with a pressure ratio that is approximately 1.89 to 1 between the pressure in supply path 106 and a pressure in inspiratory path 108. With supply path 106 producing a choked flow, the output rate (e.g., volume per second) of inspiratory path 108 may be determined based on the pressure in inspiratory path 106, which can be sensed by a pressure sensor in supply path 106, and may stay constant even if a pressure downstream from the nozzle changes. This may help ventilator 100 compensate for a variability in an input supply of air received via connection 102. By using a choked flow through supply path 106 to limit a flow into inspiratory path 108, electronic control circuitry 104 can determine the output rate of conditioned gas being delivered to inspiratory path 108 as a function of the pressure determined in supply path 106, regardless of a pressure in inspiratory path 108 or downstream from inspiratory path 108.

Pressure sensor 114 can have any suitable configuration, such as, but not limited to, a pressure transducer, a pressure gauge, or the like. Pressure sensor 114 can be configured to generate an output indicative of any suitable pressure measurement, such as, but not limited to, an absolute pressure and/or a differential pressure.

From the output rate, electronic control circuitry 104 can determine a volume of conditioned gas being delivered to a patient for a respiratory cycle. To increase the volume of conditioned gas for a future respiratory cycle, electronic control circuitry 104 can increase the amount of time which inspiratory valve 110 remains open. To decrease the volume of conditioned gas for a future respiratory cycle, electronic control circuitry can decrease the amount of time which inspiratory valve 110 remains open.

A user of ventilator 100 may set a maximum respiratory pressure that is appropriate for a particular patient. During an inspiration period, pressure sensor 114 may determine a pressure in inspiratory path 108, and if a user adjustable pre-defined peak pressure target is reached, then electronic control circuitry 104 may cause inspiratory valve 110 to close, thus holding the pressure for the remainder of the inspiration time. In some examples, each time the peak pressure setting is hit, curtailing the inspired volume of conditioned gas from being delivered, ventilator 100 may produce a visible or audible warning, such as a flashing light or beeping, thus giving the user feedback that the desired volume of conditioned gas is not being reached.

Ventilator 100 also includes dump valve 116, which provides a mechanical backup pressure reduction mechanism in the event that the operation of pressure sensor 114, electronic control circuitry 104, and inspiratory valve 110 does not adequately reduce the pressure in inspiratory path 108, e.g., to be less than or equal to the user adjustable pre-defined peak pressure target. Dump valve 116 may, for example, be a spring valve configured to open at a specific pressure. That specific may for example be 15.75 inches of water gauge (inWG), but other valves that open at different pressures may also be used.

During expiration, air is released from the lungs of the patient and out of ventilator 100 via path 118 by opening expiratory valve 120. Electronic control circuitry 104 may synchronize the opening and closing of inspiratory valve 110 with the opening and closing of expiratory valve 120, such that both are not simultaneously open. The PEEP represents a desired residual pressure in the lung and may be specified by a user of ventilator 100 (and input to electronic control circuitry 104 using any suitable user interface mechanism). Electronic control circuitry 104 may cause expiratory valve 120 to close when the pressure has decayed to the PEEP. Electronic control circuitry 104 may cause expiratory valve 120 to open multiple times to bring the pressure to the desired PEEP level. If the PEEP level is reached before the end of an expiratory period, then electronic control circuitry 104 may hold expiratory valve 120 closed for the remaining time of the expiratory period.

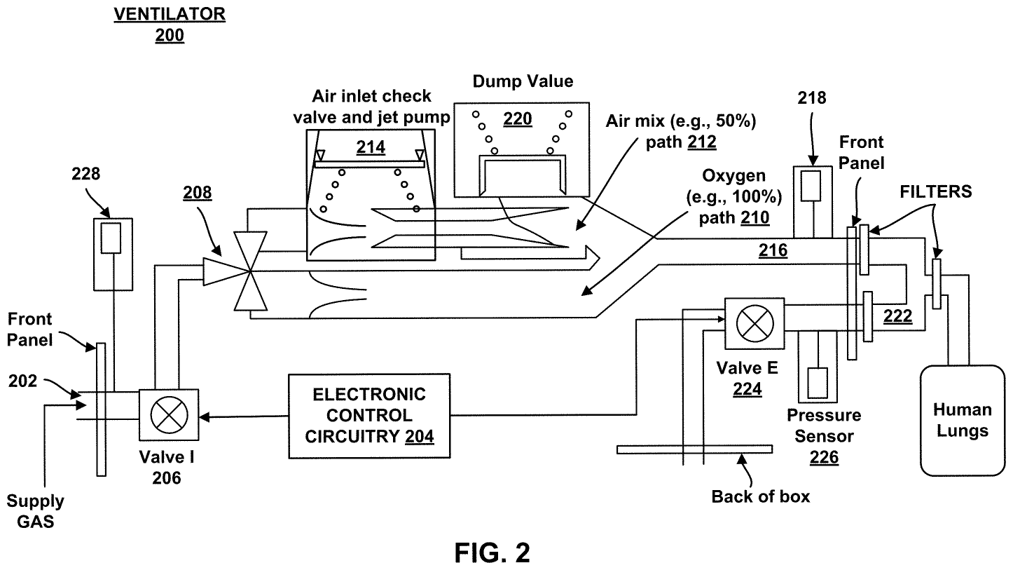

FIG. 2 shows another conceptual diagram of an example ventilator in accordance with the techniques of this disclosure. Ventilator 200 includes pneumatic connection 202, electronic control circuitry 204, inspiratory valve 206 (Valve I 206 in FIG. 2), 3-port valve 208, path 210, path 212, air inlet check valve and jet pump arrangement 214, path 216, pressure sensor 218, dump valve 220, path 222, expiratory valve 224 (Valve E 208 in FIG. 2), and pressure sensor 226. Ventilator 200 is an example of ventilator 100 of FIG. 1.

Pneumatic connection 202 can be configured to receive supply gas from a gas supply. A user, such as a doctor, nurse, or other medical clinician, may, for example, connect ventilator 200 to the gas supply via a pneumatic connection 202. Although FIG. 2 shows pneumatic connection 202 as being located in a front panel of ventilator 200, pneumatic connection 202 may located in any other accessible location on ventilator 200. Pneumatic connection 202 may, for example, include piping and/or tubing defining a pathway configured to guide supply gas from the gas supply into ventilator 200. This disclosure will explain the operation of ventilator 200 with reference to a gas supply that is 100% oxygen, but other gas supplies may also be used.

Electronic control circuitry 204 may include, or be coupled to, a user interface device that allows a user of ventilator 200 to set operating parameters for ventilator 200. Electronic control circuitry 204 may, for example, be a variable-frequency-variable-duty-cycle electronic control circuit that forms the basis of the control of ventilator 200. Electronic control circuitry 204 can provide the timing framework which is used to deliver conditioned gas flow to the lungs of a patient. Examples of the operating parameters that may be controlled by a user will be described in more detail below with reference to FIG. 3.

Inspiratory valve 206 is pneumatically connected to pneumatic connection 202 and configured to allow flow of the supply gas to path 216, through for example paths 210 or 212, when inspiratory valve 206 is open and block flow of the supply gas to paths 210 and 212, and thus to path 216, when inspiratory valve 206 is closed. Electronic control circuitry 204 is configured to control the opening and closing of inspiratory valve 206 at a specific frequency to regulate the amount of conditioned gas being supplied to the patient and to control a “breaths per minute” being supplied to the patient. Generally speaking, a longer open interval for inspiratory valve 206 results in ventilator 200 delivering to a patient a larger volume of conditioned gas per breath, and more open intervals (e.g., more open intervals per minute) results in ventilator 200 delivering more breaths per minute. Electronic control circuitry 204 may alter the amount of conditioned gas being supplied and the number of breaths per minute being supplied by ventilator 200 by varying the open-close pattern of inspiratory valve 206. The volume of conditioned gas being driven into the patient may be known and selected by a user.

As will be explained in more detail below, electronic control circuitry 204 may be configured to open inspiratory valve 206 for a period of time controlled by timing circuitry of electronic control circuitry 204 to achieve a desired inspiration volume. The time for which inspiratory valve 206 is held open may depend on an inlet pressure of the gas supply at pneumatic connection 202.

The supply gas that passes through inspiratory valve 206 is directed to 3-port valve 208. 3-port valve 208 includes a gas inlet configured to receive supply gas via inspiratory valve 206 and includes two outlets. 3-port valve 208 is generally configured such that all of the supply gas being received by the inlet is directed to the first outlet or all of the supply gas being received by the inlet is directed to the second outlet. The first outlet and second outlet may, for example, deliver the supply gas to a respective path of two distinct paths. In FIG. 2, path 210 is labeled as the oxygen (100%) path, and path 212 is labeled as the air mix (50%) path. These percentages are approximate, and other mixtures, i.e., other percentages of oxygen, may also be used. Although FIG. 2 shows ventilator 200 as having a 3-port valve and two paths, the techniques of this disclosure can also be extended to an N-port valve that has N−1 output ports and N−1 paths, where N is a suitable integer.

A user of ventilator 200 may provide a user input to ventilator 200 to cause ventilator 200 to provide 100% oxygen to a patient, in which case 3-port valve 208 directs the supply gas through path 210. In other cases, a user of ventilator 200 may provide a user input to ventilator 200 to cause ventilator 200 to provide 50% oxygen to a patient, in which case 3-port valve 208 directs the supply gas through path 212.

Path 212 includes an air inlet check valve and jet pump arrangement 214. The air inlet check valve is configured to add ambient air to the supply gas to produce the conditioned gas. Air inlet check valve and jet pump arrangement 214 allows the high pressure gas flowing out of 3-port valve 208 to create a relatively low static pressure that draws in air through the check valve. Thus, in path 212, the gas coming from the connected gas supply is mixed with ambient air, which in most common use scenarios, produces conditioned gas that has less oxygen than the gas coming from the gas supply.

3-port valve 208 pneumatically connects the supply gas to path 216 through either path 210 or path 212. Regardless of whether 3-port valve 208 directs the supply gas to path 210 or 212, the gas ultimately is routed to path 216, and path 216 delivers conditioned gas to the lungs of a patient during respiration. Path 216 includes pressure sensor 218 configured to measure a pressure of the conditioned gas being delivered to the lungs of a patient, e.g., as described with reference to pressure sensor 114 of FIG. 1.

By interacting with a user interface of ventilator 200, a user of ventilator 200 may set a maximum respiratory pressure that is appropriate for a particular patient, which electronic control circuitry 204 can determine using any suitable technique. During an inspiration period, pressure sensor 218 may determine a pressure in path 216, and if a user adjustable pre-defined peak pressure target is reached, then electronic control circuitry 204 may cause inspiratory valve 206 to close, thus holding the pressure for the remainder of the inspiration time. In some examples, each time the peak pressure setting is hit, curtailing the inspired volume of conditioned gas from being delivered, ventilator 200 may produce a visible or audible warning, such as a flashing light or beeping, thus giving the user feedback that the desired volume of conditioned gas is not being reached.

Ventilator 200 also includes dump valve 220, which provides a mechanical backup pressure reduction mechanism in the event that the operation of pressure sensor 218, electronic control circuitry 204, and inspiratory valve 206 does not adequately reduce the pressure in path 216. Dump valve 220 may, for example, be a spring valve configured to open at a specific pressure. In some examples, that specific pressure is 15.75 inWG, but other valves that open at different pressures may also be used.

Both path 210 and path 212 may have outlet nozzles leading into path 216 that are sized such that when ventilator 200 is connected to the supply gas, a pressure ratio between path 210 or path 212 and the inspiratory path (path 216) results in a choked flow of the conditioned gas through the outlet nozzle of path 210 or 212. That is, each of path 210 and path 212 include outlet nozzles that are sufficiently small, such that a pressure ratio across the nozzles results in a choked flow through the nozzles. This choked flow may be achieved, for example, with a pressure ratio that is approximately 1.89 to 1 between the pressure at three-port valve 208 and path 216. With paths 210 and 212 producing a choked flow, the output rate (e.g., volume per second) of paths 210 and 212 may be determined based on the pressure measured at pressure sensor 228 and independent of the pressure in path 216 or downstream of path 216.

As a result of the choked flow, pressure sensor 228 can be configured to determine a volume of air delivered via path 216. By using a choked flow through paths 210 and 212 to limit a flow into path 216, electronic control circuitry 204 can determine the output rate of conditioned gas being delivered to path 216 as a function of the pressure determined by pressure sensor 228 regardless of a pressure downstream from path 216.

Pressure sensor 228 may be configured to measure the pressure at the very inlet to the ventilator 200 (e.g., pneumatic connection 202), which enables ventilator 200 to account for supply pressure variation, and thus enables ventilator 200 to be used for a variety of different supply pressures. As paths 210 and 212 include choking venturis, then knowing the upstream pressure at pressure sensor 228, allows electronic control circuitry 204 to determine flow. Pressure sensor 228 enables ventilator 200 to account for, at the time of manufacture, mechanical tolerances of the components downstream by having electronic control circuitry 204 apply a gain adjustment to account for the flow restrictions caused by inspiratory valve 206, 3-port valve 208, and the interconnecting pipework which might vary from unit to unit due to manufacturing tolerances. Also, the choking flows of paths 210 and 212 make ventilator 200 naturally insensitive to mechanical tolerances of the components downstream of the choking nozzles, further reducing cost and improving accuracy and reliability.

From the output rate, electronic control circuitry 204 can determine a volume of conditioned gas being delivered to a patient for a respiratory cycle. That is, electronic control circuitry 204 can be configured to control an opening and closing of inspiratory valve 206 based on the pressure measured by pressure sensor 228. To increase the volume of conditioned gas for a future respiratory cycle, electronic control circuitry 204 can increase the amount of time which inspiratory valve 206 remains open. To decrease the volume of conditioned gas for a future respiratory cycle, electronic control circuitry can decrease the amount of time which inspiratory valve 206 remains open.

Path 222 includes pressure sensor 226 configured to measure the pressure of a fluid (e.g., air) in path 222, which generally correlates to an expiratory pressure of the lungs of the patient. A minimum pressure at which expiration flow ceases is referred to as the PEEP. Electronic control circuitry 204 may be configured to control the opening and closing of expiratory valve 224 based on a measured pressure obtained from pressure sensor 226, such that a pressure within path 222 does not fall below a desired PEEP. The PEEP may, for example, be a value set by a user of ventilator 200 via a user interface of ventilator 200.

During expiration, air is released from the lungs of a patient and out of ventilator 200 via path 222 by opening expiratory valve 224. Electronic control circuitry 204 may synchronize the opening and closing of inspiratory valve 206 with the opening and closing of expiratory valve 224, such that both are not simultaneously open. The PEEP represents a desired residual pressure in the lung and may be specified by a user of ventilator 200. Electronic control circuitry 204 may cause expiratory valve 224 to close when the pressure has decayed to the PEEP. Electronic control circuitry 204 may cause expiratory valve 224 to open multiple times to bring the pressure in path 222 to the desired PEEP level. If the PEEP level is reached before the end of an expiratory period, then electronic control circuitry 204 may hold expiratory valve 224 closed for the remaining time of the expiratory period.

In some examples of ventilator 200 of FIG. 2, inspiratory valve 206 and expiratory valve 224 may each be pneumatic solenoid valves. Inspiratory valve 206 and expiratory valve 224 may, for example, be low cost off the shelf open/shut direct acting solenoids rather than more expensive proportioning solenoids. That is, inspiratory valve 206 and expiratory valve 224 may be solenoids that are configured to transition between a fully closed state and a fully open state without maintaining any sort of persistent partially shut and partially open state. Other types of valves may be used in other examples.

In FIG. 2, paths 210, 212, 216, and 222, as well as other paths described herein, each represent pneumatic paths that may be formed by some combination of tubes, pipes, inlets, outlets, valves, connectors, regulators, filters, and other such components. Paths 210, 212, and 216 may be implemented as part of an assembly specifically designed to minimize flow resistance and noise.

Ventilator 200 may also be configured to sense a depression in the expiratory pressure and automatically trigger a new inspiratory cycle if the patient attempts to take an early breath. For example, pressure sensor 226 can be configured to detect a depression, by a specified amount below ambient pressure, in pressure in path 222. Based on the detected depression, electronic control circuitry 204 can determine that the patient is demanding a new inspiratory cycle, and based on this feedback, automatically trigger a new inspiratory cycle.

FIG. 3 shows an example of user interface of a ventilator with which a user may interact with to set the operating parameters for a ventilator, such as ventilator 100 or 200 described above. The user interface, referred to herein in some examples as controls, of FIG. 3 may, for example, be a part of or be communicatively coupled to electronic control circuitry 204 of ventilator 200. A ventilator of this disclosure may include an inspiratory-to-expiratory (I:E) ratio controller 302, which a user may use to select a ratio of an inspiration period to an expiratory period. If I:E ratio controller 302 is set to 1:1, then the duration of an inspiration period would be approximately equal to a duration of an expiratory period. If I:E ratio controller 302 is set to 1:2, then the duration of an inspiration period would be approximately half that of an expiratory period. Although, I:E ratio controller 302 is shown in FIG. 3 as having two selectable options, more options, such as options between 1:1 and 1:2 could also be included.

A ventilator of this disclosure may also include air mix controller 304 that a user may interact with to determine the amount of oxygen in the conditioned gas being delivered by the ventilator. In the example of FIG. 3, air mix controller 304 allows a user to select between a 100% oxygen ratio or a 50% oxygen ratio. Referring back to ventilator 200 of FIG. 2, a user selecting the 100% oxygen ratio may cause electronic control circuitry 204 to configure 3-port valve 208 to direct supply gas to path 210, and a user selecting 50% oxygen on air mixing path 212 may cause electronic control circuitry 204 to configure 3-port valve 208 to direct supply gas to path 212.

A ventilator of this disclosure may include Peak/PEEP control 306. Peak/PEEP controller 306 may allow a user to select both a peak pressure and a PEEP with one input, with the peak pressure always being a fixed increment above the PEEP regardless of setting. Combining peak pressure and PEEP settings into one controller 306 may reduce the number of settings a user may need to select to set-up the ventilator for a patient, which may help reduce the knowledge required to use the ventilator. In the example of FIG. 3, the fixed increment is 15 centimeters of water gauge (cmH2O), although other fixed increments may also be used. In the example of FIG. 3, a user may select a peak pressure from between 20 cmH2O and 35 cmH2O and a PEEP of 5 cmH2O and 20 cmH2O. Thus, in the example of FIG. 3, at a peak pressure of 20 cmH2O, the PEEP is 5 cmH2O. At a peak pressure of 35 cmH2O, the PEEP is 20 cmH2O.

The predetermined fixed pressure differential between the peak and PEEP pressure settings available for the ventilator may reduce the number of patients the ventilator may be used with. In some cases, a clinic may have available a plurality of different ventilators configured to have different predetermined fixed pressure differentials between the peak and PEEP pressure settings and a user can select a ventilator to use from the plurality of ventilators depending on the peak and PEEP pressure differential that is determined to be most appropriate for the particular patient.

A ventilator of this disclosure may include breaths per minute (BPM) control 308. In the example of FIG. 3, BPM control 308 may be set to values ranging from 10 to 30. A ventilator of this disclosure may also include tidal volume control 310. In the example of FIG. 3, tidal volume control 310 may be set to values ranging from 350 to 450 milliliters per inspiration. Other ranges of BPM and/or tidal volumes can be used in other examples.

A ventilator of this disclosure may also include one or more display items 312. In the example of FIG. 3, display items 312 include a visual indication of the peak pressure and the PEEP (PS in FIG. 3), as well as an indication of a sensed, i.e., instantaneous, expiratory pressure (EP in FIG. 3).

Referring back to ventilator 200 of FIG. 2, electronic control circuitry 204 may control an opening duration and opening frequency of inspiratory valve 206 and an opening duration and opening frequency of expiratory valve 224 to achieve the desired I:E ratio, BPM, and tidal volume set by the user.

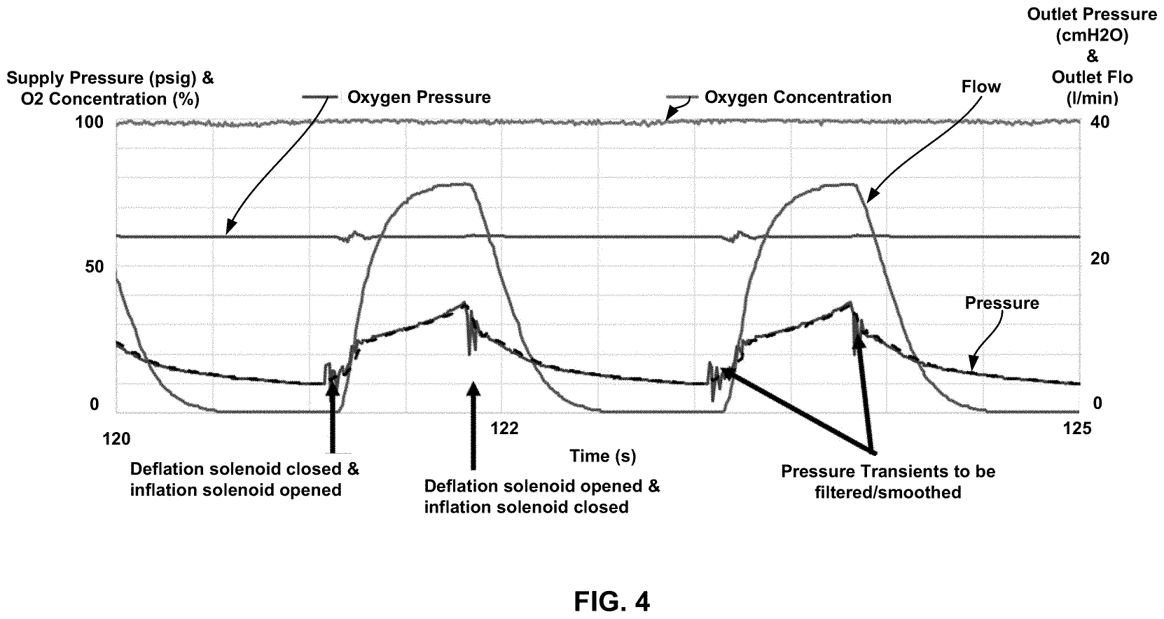

FIG. 4 shows a graphical representation of an example of an inspiratory and expiratory cycle for a specific set of operating parameters. The example of FIG. 4 shows the operation of a ventilator, as described in this disclosure. The ventilator is operating in a 100% oxygen mode with an I:E ratio of 1:2. The inlet oxygen pressure is approximately 60 psig. The ventilator is operating with a PEEP of approximately 4 cmH2O and a peak pressure of 20 cmH2O.

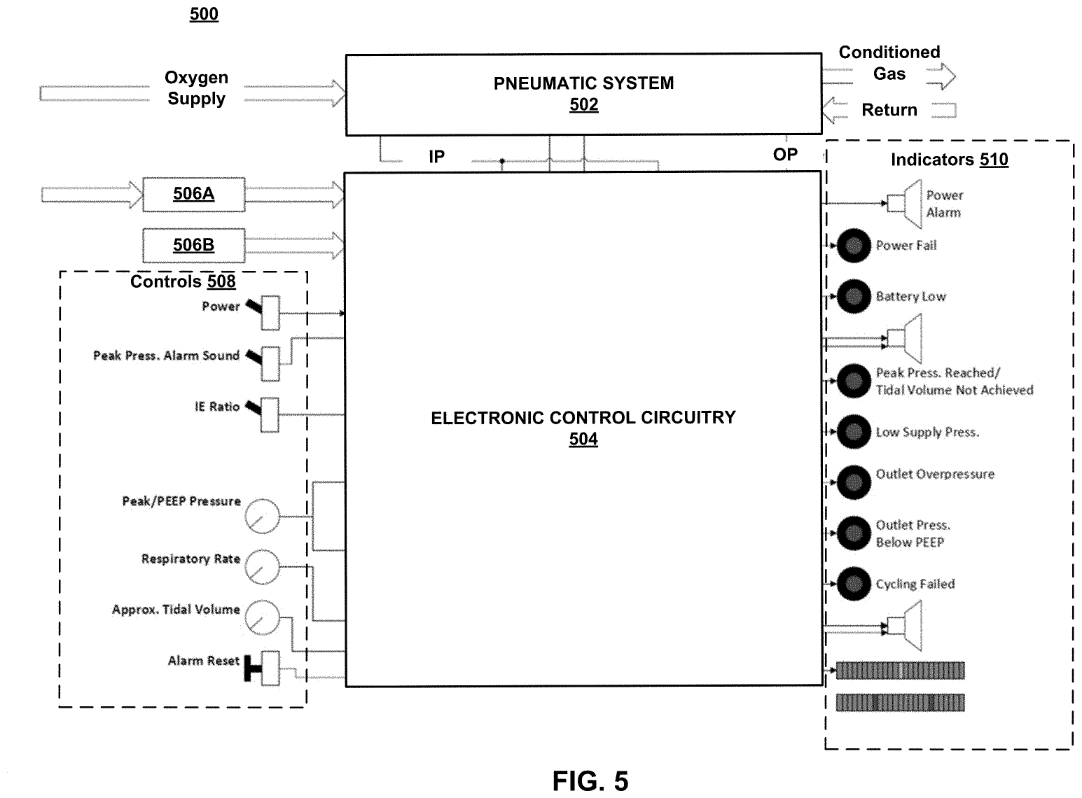

FIG. 5 shows a conceptual diagram of an example ventilator 500 in accordance with the techniques of this disclosure. FIG. 5 shows an example electronic architecture and electronic control of ventilator 200 of FIG. 2. Among other features, FIG. 5 shows examples of user inputs, user outputs, warning indicators, and conditions that may trigger those warning indicators.

Ventilator 500 includes pneumatic system 502, electronic control circuitry 504, power supplies 506A and 506B, controls 508, and indicators 510. Pneumatic system 502 may, for example, include an arrangement of valves and paths as described above with respect to FIGS. 1, 2, and elsewhere. Pneumatic system 502 may, for example, be configured to receive supply gas (e.g., an oxygen supply) and produce a conditioned gas to deliver to a patient.

Electronic control circuitry 504 may implement functionality described above with respect to electronic control circuitry 104, 204, or elsewhere. Electronic control circuitry 504 may, for example, include power supply electronics, an electronic controller, control electronics, and monitoring electronics. The control electronics may, for example, control the tidal volume and other operations of ventilator 500 based on an inlet pressure (IP) and an outlet pressure (OP) as described above with respect to FIGS. 1 and 2.

Ventilator 500 may be configured to receive power from one or both of power supplies 506A and 506B. Power supply 506A may, for example, represent a mains power supply, such as a 120V or 230V wall power. Power supply 506B may be a backup, or alternative, power source such as a battery, that can be used when power supply 506A is unavailable.

Controls 508 represent user controls for a clinician or other user of ventilator 500 to set operating parameters for ventilator 500. Controls 508 may, for example, include a power toggle switch for turning ventilator 500 on and off, a peak pressure toggle switch for turning a peak pressure alarm on or off, and an I:E ratio toggle switch that operates similarly to I:E ratio controller 302 described above. Controls 508 may also include dials for allowing the user to select a peak/PEEP pressure, a respiratory rate, and an approximate tidal volume, e.g., as described with reference to FIG. 3. Controls 508 may also include an alarm reset button for silencing or resetting any of the alarms or other indicators of indicators 510.

Indicators 510 represent indicators that may present information to a clinician or other user of ventilator 500. Indicators 510 may, for example, be configured to present information that is detected by monitoring electronics within electronic control circuitry 504. Indicators 510 may, for example, include light emitting diodes (LEDs) configured to be illuminated by control circuitry 504 to indicate conditions such as a power failure, a low battery, peak pressure being exceeded or a tidal volume not being achieved, a low supply pressure, an outlet overpressure, an outlet pressure below a PEEP, a cycle failure, or any combination thereof. In some examples, indicators 510 may also include sound generating circuitry configured to generate one or more audible alarms that can indicate the conditions identified above or other conditions. Indicators 510 may also include non-binary visual indicators that can use multiple LEDs to indicate, for example, a peak pressure and a PEEP, as well as an indication of a sensed, i.e., instantaneous, expiratory pressure.

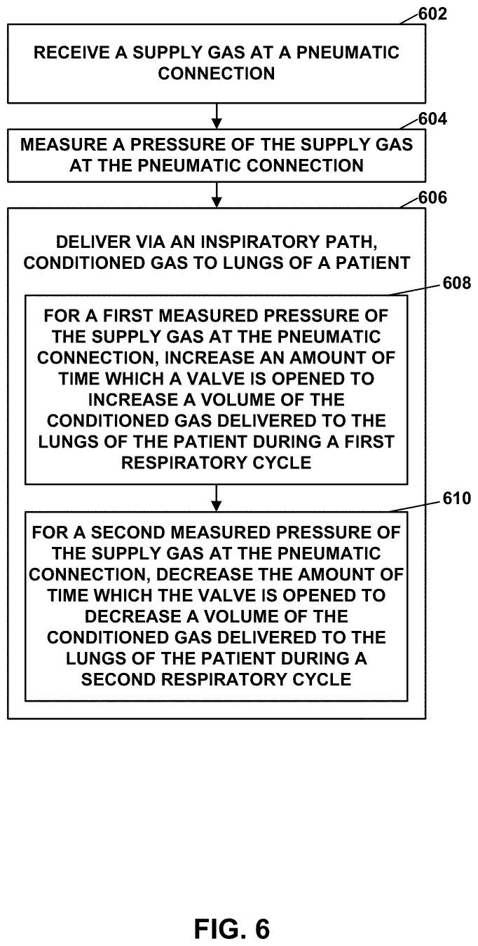

FIG. 6 represents an example process that can be performed by a ventilator of this disclosure, such as ventilator 100, ventilator 200, ventilator 500, or another ventilator contemplated by this disclosure. In the example of FIG. 6, the ventilator receives a supply gas at a pneumatic connection (602). The ventilator (e.g., control circuitry) measures a pressure of the supply gas at the pneumatic connection (604). The ventilator delivers, via an inspiratory path, conditioned gas to lungs of a patient (606). To deliver the conditioned gas to the lungs of the patient, for a first measured pressure of the supply gas at the pneumatic connection, the ventilator increases an amount of time which a valve is opened to increase a volume of the conditioned gas delivered to the lungs of the patient during a first respiratory cycle (608), and for a second measured pressure of the supply gas at the pneumatic connection different from the first measure, the ventilator decreases the amount of time which the valve is opened to decrease a volume of the conditioned gas delivered to the lungs of the patient during a second respiratory cycle (610).

The ventilator may, for example, deliver the supply gas to the inspiratory path, via an air mixing path that adds ambient air to the supply gas to produce the conditioned gas or deliver the supply gas to the inspiratory path, via a non-mixing path. The air mixing path may include an outlet nozzle sized such that while receiving the supply gas, a pressure ratio between the air mixing path and the inspiratory path results in a choked flow of the conditioned gas through the outlet nozzle. Similarly, the non-mixing path may include an outlet nozzle sized such that while receiving the supply gas, a pressure ratio between the non-mixing path and the inspiratory path results in a choked flow of the conditioned gas through the outlet nozzle.

The term “circuitry” as used herein, including control circuitry 104, 204, and 504, may refer to any of the foregoing structure or any other structure suitable for processing program code and/or data or otherwise implementing the techniques described herein. Circuitry may, for example, include any of a variety of types of solid state circuit elements, such as central processing units (CPUs), CPU cores, digital signal processors (DSPs), application specific integrated circuits (ASICs), mixed-signal integrated circuits, filed-programmable gate arrays (FPGAs), microcontrollers, programmable logic controllers (PLCs), programmable logic device (PLDs), complex PLDs (CPLDs), systems on a chip (SoC), any subsection of any of the above, an interconnected or distributed combination of any of the above, or any other integrated or discrete logic circuitry, or any other type of component or one or more components capable of being configured in accordance with any of the examples disclosed herein.

As used in this disclosure, circuitry may also include one or more memory devices, such as any volatile or non-volatile media, such as a RAM, ROM, non-volatile RAM (NVRAM), electrically erasable programmable ROM (EEPROM), flash memory, and the like. The one or more memory devices may store computer-readable instructions that, when executed or processed the circuitry, cause the circuitry to implement the techniques attributed herein to circuitry. The circuitry of this disclosure may be programmed, or otherwise controlled, with various forms of firmware and/or software.

The techniques described above may enable ventilators of this disclosure (e.g., ventilators 100, 200, and 500) to use primarily, or even only, analog circuitry. Most existing ventilators use digital circuitry with complex hardware and software. Complex digital hardware and software can involve very long and expensive development and verification processes for safety related items such as ventilators. Analog circuitry can be more reliable, easier to ruggedize, and cheaper to fabricate without the need for expensive test equipment and highly skilled labor. It may also be easier to verify that the functionality of analog circuitry works correctly in safety related applications. Analog circuitry can also be easier to test and repair. Furthermore, relatively simple analog circuitry can interface directly to a wide variety of less expensive analog output pressure sensors, such that the ventilator could be produced without reliance on complex supply chains and using sensors available from multiple sources.

Various illustrative aspects of the disclosure have been described above. These and other aspects are within the scope of the following claims.

What is claimed is:

1. A ventilator comprising:
a pneumatic connection configured to receive supply gas;
an inspiratory path configured to deliver conditioned gas to lungs of a patient;
a valve pneumatically connected to the pneumatic connection and configured to allow flow of the supply gas to the inspiratory path when the valve is open and block flow of the supply gas to the inspiratory path when the valve is closed;
a pressure sensor configured to measure a pressure of the supply gas at the pneumatic connection; and
electronic control circuitry configured to control an opening and closing of the valve based on the measured pressure to produce a desired volume of conditioned gas in the inspiratory path.

2. The ventilator of claim 1, wherein the supply gas is pneumatically connected to the inspiratory path via an air mixing path that includes an air inlet configured to add ambient air to the supply gas to produce the conditioned gas.

3. The ventilator of claim 2, wherein the air mixing path includes an outlet nozzle, wherein the outlet nozzle is sized such that when the ventilator is connected to the supply gas, a pressure ratio between the air mixing path and the inspiratory path results in a choked flow of the conditioned gas through the outlet nozzle.

4. The ventilator of claim 1, wherein the supply gas is pneumatically connected to the inspiratory path via a non-mixing path configured to deliver the supply gas to the inspiratory path.

5. The ventilator of claim 4, wherein the non-mixing path includes an outlet nozzle, wherein the outlet nozzle is sized such that when the ventilator is connected to the supply gas, a pressure ratio between the non-mixing path and the inspiratory path results in a choked flow of the supply gas through the outlet nozzle.

6. The ventilator of claim 1, wherein the pressure sensor comprises a first pressure sensor, the ventilator further comprising:
an expiratory path configured to remove exhaled air from the lungs of the patient via a second valve; and
a second pressure sensor configured to measure a pressure of air in the expiratory path,
wherein the electronic control circuitry is configured to control an opening and closing of the second valve to prevent the measured pressure of air in the expiratory path from falling below a minimum pressure.

7. The ventilator of claim 1, further comprising:
a user interface configured to set a peak pressure for the ventilator and a positive end-expiratory pressure (PEEP), wherein the peak pressure and the PEEP differ by a fixed amount across a range of peak pressure values and PEEP values.

8. The ventilator of claim 1, further comprising:
a user interface configured to set a peak pressure for the ventilator and a positive end-expiratory pressure (PEEP), wherein a value for the PEEP is constrained to be a fixed amount different from the peak pressure.

9. The ventilator of claim 1, wherein to increase a volume of the conditioned gas delivered to the lungs of the patient during a respiratory cycle, the electronic control circuitry is configured to increase an amount of time which the valve is opened.

10. The ventilator of claim 1, wherein to decrease a volume of the conditioned gas delivered to the lungs of the patient during a respiratory cycle, the electronic control circuitry is configured to decrease an amount of time which the valve is opened.

11. The ventilator of claim 1, further comprising:
a selector valve, wherein in a first position, the selector valve pneumatically connects the supply gas to the inspiratory path via an air mixing path that includes an air inlet configured to add ambient air to the supply gas to produce the conditioned gas, and wherein in a second position, the selector value pneumatically connects the supply gas to the inspiratory path via a non-mixing path to produce the conditioned gas.

12. The ventilator of claim 11, wherein the air mixing path includes a first outlet nozzle, wherein the first outlet nozzle is sized such that when the ventilator is connected to the supply gas, a first pressure ratio between the air mixing path and the inspiratory path results in a first choked flow of the conditioned gas through the first outlet nozzle, and wherein the non-mixing path includes a second outlet nozzle, wherein the second outlet nozzle is sized such that when the ventilator is connected to the supply gas, a pressure ratio between the non-mixing path and the inspiratory path results in a second choked flow of the supply gas through the second outlet nozzle.

13. The ventilator of claim 11, further comprising:
a user interface for causing the selector valve to switch between the first position and the second position.

14. The ventilator of claim 11, wherein the selector valve comprises a 3-port valve.

15. The ventilator of claim 11, wherein the selector valve comprises a solenoid valve.

16. The ventilator of claim 11, wherein when the selector valve is in the second position, the conditioned gas is the same as the supply gas.

17. The ventilator of claim 1, wherein the supply gas comprises oxygen.

18. A method comprising:
receiving a supply gas at a pneumatic connection;
measuring a pressure of the supply gas at the pneumatic connection; and
delivering, via an inspiratory path, conditioned gas to lungs of a patient, wherein delivering the conditioned gas to the lungs of the patient comprises:
for a first measured pressure of the supply gas at the pneumatic connection, increasing an amount of time which a valve is opened to increase a volume of the conditioned gas delivered to the lungs of the patient during a first respiratory cycle; and
for a second measured pressure of the supply gas at the pneumatic connection, decreasing the amount of time which the valve is opened to decrease a volume of the conditioned gas delivered to the lungs of the patient during a second respiratory cycle.

19. The method of claim 18, further comprising:
delivering the supply gas to the inspiratory path, via an air mixing path that adds ambient air to the supply gas to produce the conditioned gas, wherein the air mixing path comprise an outlet nozzle sized such that while receiving the supply gas, a pressure ratio between the air mixing path and the inspiratory path results in a choked flow of the conditioned gas through the outlet nozzle.

20. The method of claim 18, further comprising:
delivering the supply gas to the inspiratory path, via a non-mixing path, wherein the non-mixing path comprise an outlet nozzle sized such that while receiving the supply gas, a pressure ratio between the non-mixing path and the inspiratory path results in a choked flow of the conditioned gas through the outlet nozzle.
